(19) United States
(12) Patent Application Publication
(10) Pub. No.: US 2022/0396753 A1
(43) Pub. Date: Dec. 15, 2022

(54) BIOREACTOR APPARATUS AND METHOD FOR IN-VITRO HEART SIMULATION

(71) Applicant: Imperial College Innovations Limited, London (GB)

(72) Inventors: Fotios Pitoulis, London (GB); Cesare Terracciano, London (GB); Pieter De Tombe, London (GB)

(73) Assignee: Imperial College Innovations Limited, London (GB)

(21) Appl. No.: 17/775,641

(22) PCT Filed: Nov. 11, 2020
(86) PCT No.: PCT/GB2020/052869

§ 371 Date: May 10, 2022

(30) Foreign Application Priority Data
Nov. 11, 2019 (GB) 1916385.6

(51) Int. Cl. C12M 3/00; C12M 1/42; C12M 1/34; C12M 1/02; C12N 13/00

(52) U.S. Cl. CPC C12M 21/08; C12M 35/04; C12M 41/00; C12M 41/24; C12M 35/02; C12N 13/00

(57) ABSTRACT

An apparatus comprising an actuator for moving an actuator rod and a bioreactor vessel is disclosed herein. The bioreactor vessel comprises a container for holding a liquid, a mounting for mounting a tissue sample in the container, and, an actuator coupling to enable the actuator rod to be connected for applying mechanical force to the tissue sample. The apparatus also comprises a seat, fixed with respect to the actuator and configured for locating the reactor vessel in a location selected so that the actuator can be connected for applying said force via the actuator coupling. The reactor vessel is removable from the apparatus.

### FIELD OF INVENTION

The present invention relates to methods and apparatus for in vitro study of tissue samples, such as bioreactors, and more particularly to the structure of such apparatus, the construction thereof, and methods of assembling and using such apparatus.

### BACKGROUND

Cardiovascular research uses in vivo or in vitro heart models to understand the normal and diseased operations of the heart. In vivo research is expensive, low-throughput, and ethically questionable. Additionally, studying the heart in vivo is complicated by multiple, dynamically interacting systems and organs, which make mapping of cause-effect relationships very difficult. In vitro models solve the complexity problem by studying the heart in isolation. In vitro heart models are often studied in artificial conditions, meaning that they can only be reliably studied in the short-term. This is because when keeping heart tissue in an artificial environment for prolonged periods of time (culture) the properties of the tissue change dramatically to the extent that they have minimal resemblance to the in vivo heart. This can lead to artefacts and false-positive/negative findings.

The fundamental operation of the heart is to displace blood from its chambers to peripheral tissues to maintain oxygen and nutrient supply. Therefore, any heart model to be used for translational research should at minimum attempt to recapitulate this.

Myocardial Slices (MS) have been used for this purpose in basic mechanical stretching protocols which include stretching a MS to a desired length and then stimulating it with electrodes to beat. However, such protocols are crude and minimise the relevance of the model being studied and its ability to translate findings from bench to bedside.

### SUMMARY

Aspects and embodiments of the present disclosure are set out in the appended claims, and may provide an improved biological model for cardiovascular research, known as myocardial slices (MS).

An aspect of the disclosure provides an apparatus comprising:
an actuator for moving an actuator rod;
a bioreactor vessel comprising:
   a container for holding a liquid;
   and, an actuator coupling to enable the actuator rod to be connected for applying mechanical force to a tissue sample mounted in the container;
a seat, fixed with respect to the actuator and configured for locating the reactor vessel in a location selected so that the actuator can be connected for applying said force to said mounting via the actuator coupling.

The bioreactor vessel may be removable from the apparatus. For example the container may have a base shaped to cooperate with the seat to hold the container in a fixed position in the seat. For example, the two may fit snugly together so as to prevent lateral (horizontal) movement of the vessel (e.g. it may fix its position to within a tolerance of better than ±1.5 mm, for example better than ±1 mm, for example better than ±0.5 mm.

The seat may comprise a heat provider, such as thermoelectric pad. The base of the reactor vessel may be thermally conductive, and the walls of the container may be thermally insulating. For example the base may comprise a metal such as stainless steel and the walls may comprise a polymer such as PEEK.

The heat provider may be configured to control the heat provided to the thermally conductive base based on a sensor signal obtained from a liquid in or flowing through the container. The sensor signal is obtained from a sensor disposed for sensing the temperature of liquid in a liquid recirculation system of the apparatus.

The container may comprise an inlet for flow of liquid into the container from the recirculation system, and an outlet for the flow of liquid out of the container into the recirculation system. The inlet and/or the outlet may comprise releasable and self-sealing couplings such as an elastically deformable grommet and/or a screw thread, or other quick disconnect feature. This can assist in simple removal of the bioreactor vessel from the apparatus and its subsequent reconnection.

The apparatus may comprise a sensor, such as a force transducer, for sensing mechanical force. This mechanical force sensor may be arranged for sensing mechanical force generated by the tissue sample. For example, the sensor may be connected to the mounting, which may be movably disposed in the container and arranged for movement by the actuator. The sensor may be arranged so that when the bioreactor vessel is held in the seat, the sensor can be connected to the mounting for sensing movement (e.g. force, speed, or displacement) of said mounting. The sensor may be connected to the mounting by a sensor rod 22. The sensor and the actuator may be connected to separate parts of the mounting each arranged to hold opposite edges of a tissue sample, so that when a tissue sample is held in the mounting the two parts of the mounting are joined together by the tissue sample.

The apparatus may comprise a controller configured to control the actuator based on a sensor signal obtained from this sensor. The senor signal may indicate force and/or movement caused by the mechanical response of the tissue sample to a stimulus, such as an electric stimulus.

The apparatus may be configured to provide a cyclic (e.g. repeating, e.g. periodic) stimulus to the tissue sample wherein the control of the actuator during a cycle of the stimulus is based on sensing performed in a preceding cycle of the stimulus.

The actuator coupling may be arranged in the wall of the container so that, when the reactor vessel is located in said location in the seat, an actuator rod can connect the actuator to the tissue sample. For example the coupling may comprise an aperture to allow the actuator rod to pass therethrough, but other types of coupling may be used (e.g. mechanical linkages and/or electromechanical means).

The apparatus may further comprise the actuator rod, and or the sensor rod 22 for joining the mounting to the actuator or sensor respectively. These rods may be removable from the actuator.

The rods may be adapted for disinfection in an auto-clave. The reactor vessel may also be adapted for disinfection in an auto-clave. For example the walls of the container may comprise PEEK, or another appropriate polymer such as Ertacetal C, polycarbonate, medical grade polyethylene and similar materials. The base may comprise stainless steel (for example medical grade stainless steel—316) or another appropriate material such as titanium

The apparatus may comprise an electrical stimulus provider for providing electrical stimulus to the tissue sample and may also comprise a temperature sensor. The apparatus may also comprise a signal interface for connection to the temperature sensor and/or for connection to the electrical stimulus provider. The seat may be arranged so that when the vessel is located in said seat it is positioned for connection of the signal interface to a corresponding interface of the bioreactor vessel. For example the bioreactor vessel may comprise electrical contacts which, when the base of the vessel is held in the seat, are brought into contact with electrical contacts carried by the bioreactor vessel.

The seat and the bioreactor vessel may comprise complementary engagement features for locating the reactor vessel in a chassis of the apparatus and holding it in a fixed position on the chassis.

The reactor vessel may comprise a removable lid. The lid may be adapted for disinfection in an auto-clave, and may be transparent. For example it may comprise polycarbonate and may be clear. Other materials may also be used for the lid, such as, for example, ceramic glass, which may be temperature resistant (heat tolerant).

The apparatus may comprise a temperature sensor for sensing temperature of a liquid held in the container. This sensor may be carried by the container, or the liquid recirculation system, or it may be disposed in the seat for sensing temperature of the thermally conductive base of the container.

MS may comprise ultra-thin living sections of heart tissue, which may be less than 300 μm thick. They may comprise structure, architecture and function of the native heart tissue. A MS may comprise cell populations, extracellular matrix, and the connections between them as they would be found in the heart. In some embodiments an MS comprises all such cell populations, extracellular matrix, and connections. Thus, in contrast to other oversimplified in vitro models (e.g. isolated cells), MS merge the complexity of the heart with the indispensable experimental control and insight that an in vitro model offers.

In another aspect of the disclosure there is provided a method of providing an in vitro cardiac model, the method comprising:
applying a periodic electrical stimulus waveform to a sample of cardiac tissue; and
applying a periodic mechanical force waveform to the sample;
wherein the mechanical force waveform is synchronised with the electrical stimulus waveform;
the method further comprising:
adjusting the time period of the periodic electrical stimulus waveform; and
adjusting the mechanical force waveform based on the adjustment to the time period of the electrical stimulus waveform.

Each period of the electrical stimulus waveform may comprise a time interval during which an electric stimulus is applied to the sample, and a time interval in which no electrical stimulus is applied.

Applying the periodic mechanical force waveform may comprise cyclically moving sample of cardiac tissue between an extended state and a shortened state. Moving the sample between the extended state and the shortened state, and vice versa, may comprise respectively shortening and lengthening the distance between the holders, e.g. the mountings, between which the sample is held.

Applying a periodic mechanical force waveform that is synchronised with the electrical stimulus waveform may comprise providing a full single cycle of compression and extension of the sample between each cycle of the electrical stimulus waveform, e.g. between each application of an electrical stimulus to the tissue. It may additionally or alternatively comprise providing a constant phase shift between the electrical stimulus waveform and the mechanical force waveform, for example, the start of the time interval in which the electrical stimulus is applied to the sample may provide a fiduciary marker and the start of the mechanical waveform may have a constant time offset from this marker. In some examples the time offset may be zero.

Each period of the mechanical force waveform may comprise a static interval in which the sample is held static in the extended state, and a dynamic interval in which the sample is moved into the shortened state and moved back into the extended state. As just one example the static interval may comprise 40% of the total period of the waveform, and the dynamic interval may comprise 60%. The relative length of time of the static and dynamic intervals may vary in different examples and may be adjusted in operation. The absolute length of the static and dynamic intervals and/or of the waveform as a whole may also vary.

Adjusting the mechanical force waveform may comprise adjusting at least one of: the duration of the static interval, and the duration of the dynamic interval.

The method may further comprise obtaining an instruction signal and providing the adjustment to the period of the electrical stimulus waveform in response to the obtained instruction signal. The instruction signal may be obtained from a user interface.

The electrical stimulus is provided by a periodic electrical pulse. The periodic electrical pulse may be bipolar.

The method may comprise providing a trigger signal simultaneously upon each application of the electrical stimulus, and applying the mechanical force in response to the trigger signal.

Applying a periodic mechanical force waveform to the sample may comprise providing instructions to an actuator to shorten and/or extend the sample.

The method may comprise sensing a mechanical response of the sample. The method may further comprise adjusting at least one of the periodic electrical stimulus waveform; and the periodic mechanical force waveform based on the sensed response.

The mechanical force waveform being synchronised with the electrical stimulus waveform may comprise the start of each cycle of the periodic mechanical force waveform having a constant phase and/or timing offset from the start of each cycle of the periodic electrical stimulus waveform.

A controller configured to form any of the above methods is also provided. A computer program product comprising computer program instructions configured to program a controller to perform any of the above methods is also provided.

Embodiments aim to reproduce essential physiological functions of the heart. Thus, they may aim to avoid or reduce problems which occur in findings of reductionist in vitro systems (e.g. isolated heart cells) which often do not translate to the whole organism

Embodiments of the disclosure aim to reflect the complexity of mechanical and electrical events that occur in the heart—known as the cardiac cycle. Embodiments simulate in vivo conditions wherein following electrical activation, the heart generates pressure while changing shape (i.e. volume) in order to push blood out of the ventricles to the rest of the body. They may thus simulate the timecourse of pressure-volume changes of the cardiac cycle, for example a sequence of dynamic phases where only pressure is changing while volume is constant, vice versa, and when both are changing at the same time. This may enable study of in vitro tissue for extended periods of time, and by simulating such conditions in vitro embodiments may avoid the introduction of artificial elements into studies of heart models.

Embodiments of the disclosure aim to provide a heart model that can be studied in the long-term (i.e. culture) without changes in its archetypical properties (i.e. beating like it would in vivo). Embodiments thus aim to provide a methods and apparatus in which a model (e.g. of cardiac tissue, such as a myocardial slice, can be exposed to the normal physiological conditions of the heart. Achieving this would open frontiers with translational impact (e.g. cardiotoxicity drug assays predictive studies, novel molecular entities screening, etc.) that are currently performed in systems with minimal predictive capacity due to inherent artificiality of the model and changes occurring during culture.

Embodiments of the disclosure also aim to provide a heart model in which the electrical stimulus and mechanical force applied to the tissue are synchronised with one another, and remain synchronised over many cycles of operation.

Embodiments also aim to enable control of the mechanical expansion and compression of the tissue, such that the mechanical force applied to the tissue remains synchronised with the electrical stimulus applied, as the time period of the electrical stimulus is varied. This may enable the response of the heart to be accurately modelled across a range of heartrates. Such adjustments may occur in real time.

These and the other embodiments described herein aim to address technical problems related to those outlined above, and others described herein.

### BRIEF DESCRIPTION OF DRAWINGS

Embodiments of the disclosure will now be described in more detail, by way of example only, with reference to the accompanying drawings, in which:

FIG. 1 shows a schematic view of a bioreactor apparatus comprising a plan view illustrated in inset A, and a section view in inset B showing a section along the line B-B.

FIG. 2 shows a flow chart illustrating one mode of operation of a bioreactor apparatus such as that illustrated in FIG. 1;

FIG. 3 includes a an elevation view of a bioreactor apparatus;

FIG. 4 shows a plan view of a reactor vessel for use in a bioreactor apparatus such as that illustrated in FIG. 2; and

FIG. 5 is an exploded view of a bioreactor apparatus;

FIG. 6 gives a schematic view of a bioreactor apparatus;

FIG. 7 illustrates an example of a method that may be performed by a bioreactor apparatus;

FIG. 8 shows a graph illustrating part of a cycle of operation of a bioreactor apparatus;

FIG. 9 illustrates an example of another method that may be performed by a bioreactor apparatus.

In the drawings, like reference numerals are used to indicate like elements

### SPECIFIC DESCRIPTION

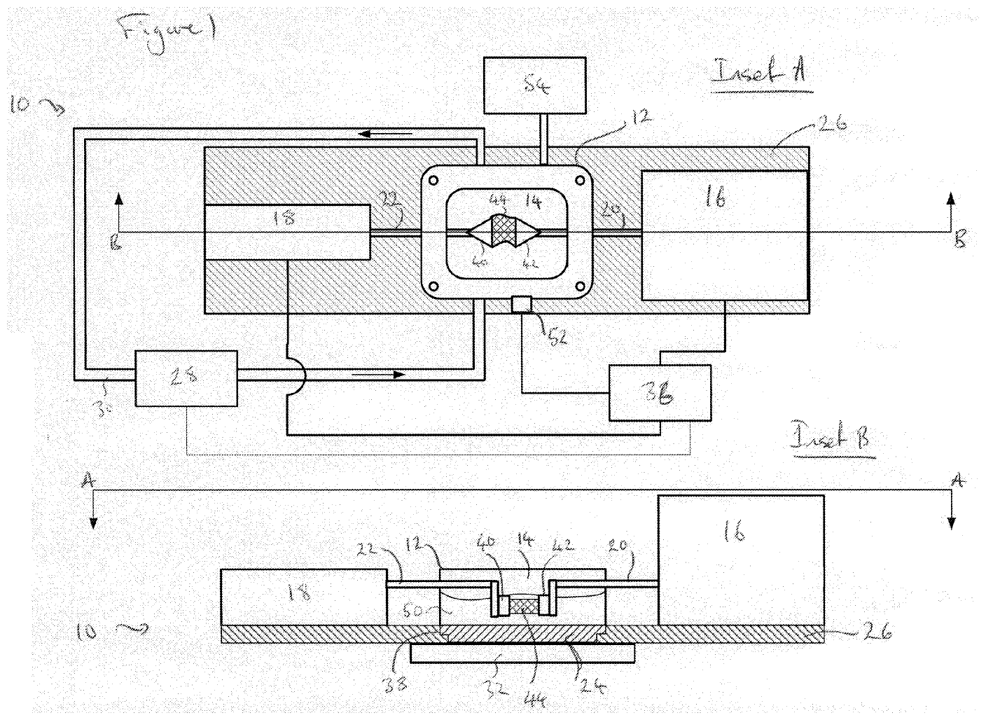

FIG. 1 is a diagram of one example of a bioreactor apparatus according to the present disclosure.

As an over view—the apparatus 10 shown in FIG. 1 comprises a bioreactor vessel. The bioreactor vessel 12 comprises a container 14 for holding a liquid 50, and arranged in the container 14 is a mounting 40, 42 for a tissue sample, such as cardiac tissue, for example a myocardial slice (MS).

The apparatus 10 also comprises an actuator 16, a force sensor 18, a chassis 26, a seat 38 carried by the chassis 26 for seating the reactor vessel 12, a liquid system 28, 30, a heat provider 32, an electrical stimulus provider 52, and a controller 36.

In the apparatus 10 shown in FIG. 1, the seat 38 is arranged so that when the reactor vessel is held by the seat 38, it is also positioned so that the actuator 16 can be connected for applying force to the tissue sample 44 in the container, e.g. by a rod. The reactor vessel is removable from the rest of the apparatus 10, e.g. as a single integrated unit, and may consist essentially of components which are adapted for cleaning in an auto clave (are “auto-clavable”). In addition the controller 36 can control operation of the actuator 16 based on the mechanical response of the tissue sample 44 to a stimulus, as sensed by the force sensor. This modular construction may simplify the use, reuse and cleaning of the apparatus 10. The adaptive nature of the stimulus and feedback provided by the tissue may provide a more realistic model of cardiac tissue. A variety of other problems may also be addressed by the present disclosure as will become apparent from the detailed discussion of this and other apparatus provided herein.

As to the structure of the apparatus 10 shown in FIG. 1, in this example, the apparatus 10 has a chassis 26 which carries the actuator, the sensor, and the seat 38 which is disposed between the sensor 18 and the actuator. The bioreactor vessel 12 is removably seated in the seat 38, and the mounting 40, 42 for the tissue sample 44 is disposed in the container 14 of the vessel.

The mounting 40, 42 comprises two parts, movable with respect to each other and arranged to hold the tissue sample 44 between them. The first part 42 of the mounting is connected to the actuator 16 by an actuator rod 20. The second part 40 of the mounting is connected to the sensor 18 by a sensor rod 22. The wall of the reactor vessel adjacent the actuator 16 comprises an aperture, to allow the actuator rod 20 to pass therethrough for coupling the first part 42 of the mounting to the actuator. The actuator rod 20 may comprise a dog-leg (e.g. one or more bends in an otherwise straight rod). Likewise, the wall of the reactor vessel adjacent the sensor 18 may also comprise an aperture, to allow the sensor rod 22 to pass therethrough for coupling the second part 40 of the mounting to this rod too may comprise a dog-leg. Thus, the apertures which enable coupling to the actuator 16 be above the level of any liquid 50 held in the container 14 and yet still ensure that the mounting 40, 42 can be at least partially submerged in that liquid 50.

The mounting 40, 42 is thus arranged so that a tissue sample 44 held on the mounting 40, 42 can be bathed in the liquid 50 in the container. The first part 42 of the mounting is for holding one edge of the tissue sample 44, and the second part 40 is for holding the other (opposite) edge of the tissue sample 44. The two parts are movable laterally with respect to each other (e.g. towards and away from each other). Thus the tissue sample 44 can relax (e.g. extend) and contract (e.g. shorten) while held securely between the two parts of the mounting 40, 42. This can allow force to be applied to the tissue sample 44 by operation of the actuator 16 moving the actuator rod 20. It can also allow the tissue sample 44 to extend and contract in response to stimulus, such as electrical stimulus. The parts of the mounting 40, 42 may be separate from each other, and may each comprise a connection point for connection to a rod, and an elongate transverse tissue holder for holding one side of the tissue sample 44.

As illustrated in FIG. 1, the actuator rod 20 is connected to the connection point of the first part 42 of the mounting, and the sensor rod 22 is connected to the connection point of the second part 40 of the mounting. The connection points may be pivotably connected to the two rods so that the mounting parts can pivot in the plane of the tissue sample 44 (e.g. a horizontal plane such as one parallel to the floor of the container). The actuator rod 20 and the sensor rod 22 may thus be less likely to place shear stress on the tissue sample 44. One way to achieve this is that the first part of the mounting 42 and the second part of the mounting 40 each be provided by a triangular member (e.g. a triangular ring) one side of such a triangular member can be affixed (e.g. glued) to one edge of the tissue sample (during preparation of the sample). A side of the other part of the mounting can then be similarly affixed to the opposite edge of the tissue sample. The dog-legged ends of the rods for connection to the actuator and sensor respectively can then be hooked into the connection point of the triangular member (e.g. inside a vertex of the triangular ring). This can allow the part of the mounting to pivot with respect to the rod, and pivoting of the tissue slice in the horizontal plane to reduce (e.g. prevent) shear stress. The mounting 40, 42 may comprise a biocompatible material and may be single-use.

These rods are typically rigid and may be releasably connected to the actuator 16 and the sensor 18 respectively, e.g. by a screw thread or similar releasable fixing means. This can simplify removal of the reactor vessel for cleaning and can enable the rods themselves also to be auto-claved with the reactor vessel.

The walls of the container 14 are thermally insulating and typically comprise an inert material such as PEEK, or any other inert material able to reduce adsorption of the drugs being tested on the tissue sample. Examples of such materials include Ertacetal C, and medical grade polyethylene. The base 24 of the container 14 comprises a thermal conductor such as a metal, e.g. stainless steel, titanium, or platinum.

As illustrated in FIG. 1, the seat 38 is disposed in the chassis 26 between the sensor 18 and the actuator 16. The base 24 of the reactor vessel is shaped to fit snugly into the seat 38 so as to positively locate the reactor vessel in a fixed location and/or with a fixed orientation relative to the sensor 18 and the actuator. Beneath the seat 38 is the heat provider 32, for providing thermal control of the liquid 50 e.g. via the thermally conductive base 24.

The liquid system comprises a tube 30 which connects a liquid inlet of the container 14 to a liquid outlet of the container. A pump 28 is connected to the tube 30 for circulating liquid from the container 14 outlet back to the container 14 inlet. For example, the pump 28 may comprise a peristaltic pump and the tube 30 may comprise at least one flexible wall portion arranged to enable the peristaltic pump 28 to move liquid through the tube 30 without contacting the liquid. The rest of the tube 30 may be less flexible than this portion and may for example be thermally insulated. The tube 30 may be connected to the container 14 inlet and/or outlet by releasable seals, to allow the tubes of the liquid system easily to be unplugged from the bioreactor vessel 12 to allow the tubes to be replaced or removed for cleaning, and to assist in easy removal of the bioreactor vessel. For example, the tubes may be fitted with male luer locks and the inlet/outlet with female luer locks (or vice versa). Other configurations can be used.

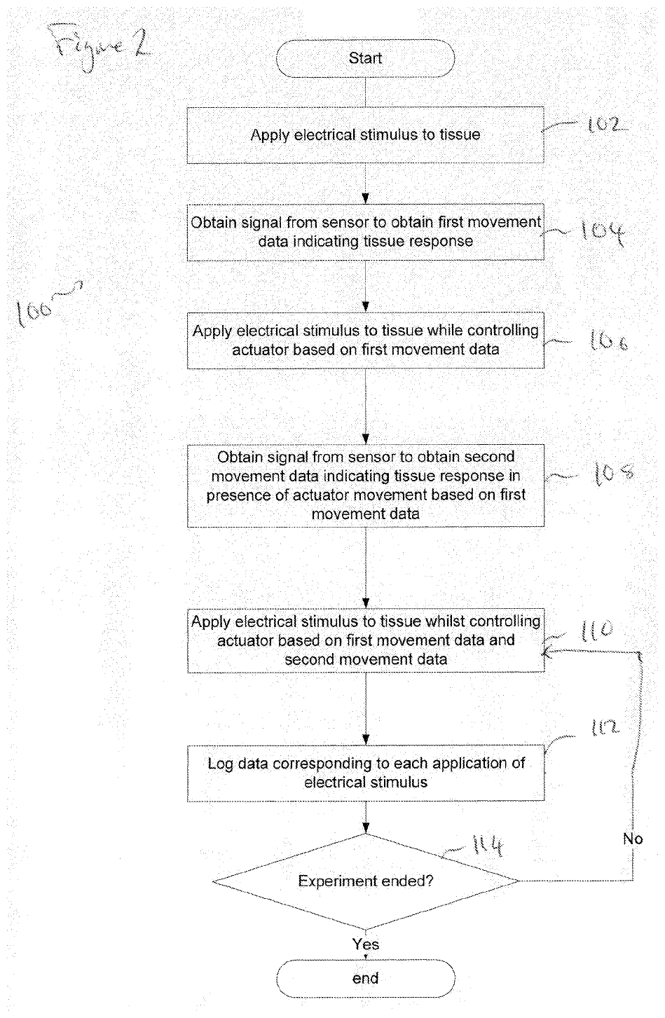

The chassis 26 of the apparatus 10 may be rigid and arranged to hold the seat 38, the sensor, and the actuator 16 in selected positions relative to each other. The positions of the sensor 18 and/or the actuator 16 may be adjustable on the chassis 26 as explained below with reference to FIGS. 2 to 4.

The apparatus 10 illustrated in FIG. 1 may also comprise an oxygen (O₂) gas supply 54, which is connected to provide O2 gas into the container, e.g. into a headspace above the liquid in the container. It may also comprise a lid 56, which covers the container 14. This, and the gas supply, may maintain a positive gas pressure inside the container 14 thereby to inhibit ingress of micro-organisms.

In the apparatus 10 shown in FIG. 1, the controller 36 is connected to the sensor, and to the actuator. It is also connected to the heat provider 32, the pump, and to the electrical stimulus provider 52.

The controller 36 may comprise processing logic and a signal interface for data acquisition and control (e.g. a DAC/ADC interface). It also comprises a data interface for providing experimental data to a computer e.g. via a communications interface such as a serial interface. Examples of the type of data that may be recorded includes:
Force generated by tissue
Actuator position
Actuator motion profile
Temperature
Fluid flow (peristaltic pump)
Tissue length
Tissue length changes during actuator motion profiles (e.g. length of tissue while it is being stretched in the ‘automatic end-point assay’ mode).

Each of the elements which the controller 36 can control via its data acquisition and control interface will now be described in turn.

The actuator 16 is configured to push and pull the actuator rod 20 for applying mechanical force to the tissue. It may have a length excursion of approximately up to 10 mm, and may have a length (displacement) resolution of 1 micron. It may have a linearity of 0.5% or better over its range, and 0.1% or better over the middle 2 mm of its range. It may comprise an electromechanical actuator such as a solenoid, or a rotary moving coil motor. The actuator may have a 20 mm total travel range, and 0.01 micron resolution, and may comprise a voice-coil actuator. Examples of suitable actuators include the 300C series of muscle levers available from Aurora Scientific of 8 Terenure Place, Terenure, Dublin, Ireland.

The sensor 18 is operable to sense force applied to the sensor 18 rod (e.g. at the second part 40 of the mounting). The sensor 18 can thus be used to sense force generated by extension or contraction of the tissue sample 44. Examples of suitable sensors include an F30 Force Transducer by Harvard Apparatus, which may have a +0.3N detectable force range, <0.1% linearity error, <0.1% hysteresis. Other appropriate sensors may be used.

The heat provider 32 may comprise a thermally conductive element disposed in the seat 38 and coupled to a heat source. For example it may comprise a heater, e.g. a thermoelectric pad, which may comprise a resistive element for joule heating or similar which may be controlled thermostatically, e.g. by the controller 36 based on a temperature signal obtained from the liquid and/or from the base 24 of the reactor vessel.

The electrical stimulus provider 52 may comprise electrical connections, such as electrodes disposed in electrical contact with the tissue sample 44, which contact may be mediated by the liquid in the vessel and/or by the actuator rod 20 or sensor rod 22. A variety of configurations are possible for delivering electrical energy to the tissue sample 44 to stimulate a response from the tissue. One example of a suitable stimulus is a constant-voltage bipolar square waveform of 0.5-40 ms duration and 0.5-100V amplitude. Frequency of pacing is usually 1 Hz but can be faster or slower depending on experiment. Typically the stimulus comprises a cyclical (e.g. repeating) electrical signal, such as a voltage.

The controller 36 may be configured to control the pump to maintain a constant flow rate of the liquid through the container 14 from inlet to outlet. The ability to provide known or constant (e.g. non-pulsatile) flow may reduce stress on the tissue, and/or may improve accuracy of force measurement because any systematic error that could otherwise be caused by that flow can be reduced or accounted for.

The controller 36 may also be configured to control the heat provider 32 based on the temperature of the liquid so as to maintain the temperature of the liquid at a temperature selected according to the type of tissue and/or the experimental protocol. For example, for human tissue, such as an MS from a human heart it may be configured to control the temperature of the liquid to be 37° C.

FIG. 2 shows a flow chart illustrating one possible mode of operation of a bioreactor apparatus, such as that shown in FIG. 1. This method includes a first calibration step in which the response of the tissue to electrical stimulus is measured, and then this first movement data obtained during this first calibration step is used to control the mounting during subsequent application of the stimulus. It may also include a second calibration step, in which second movement data is obtained by sensing the force applied to the sensor 18 by the tissue whilst also controlling the actuator 16 to move according to the first movement data. Subsequently each time a stimulus is applied during an experimental protocol, the mounting can be controlled, by the controller operating the actuator, based on the first movement data and the second movement data so as to reduce or minimise mechanical load placed on the tissue. This may also improve the accuracy of measurement.

In such experiments, the effect of the connection of the tissue sample 44 to the mounting may be reduced and longevity of the sample may be increased. The accuracy and quantity of data obtained may thus be increased as compared to prior art methods.

A variety of embodiments of this method are possible, but one example will now be described in detail.

In this example, an electrical stimulation is applied 102 to the tissue sample 44, e.g. via the electrical stimulus provider 52. This electrical stimulus may comprise a cycle, or part of a cycle, of a cyclic waveform. This stimulus may cause the tissue to respond by moving, e.g. contracting, which can generate force on the mounting which can be sensed by the sensor. Thus, during the application of electrical stimulus, and during the response of the tissue sample 44, a signal is acquired 104 from the sensor 18 indicating the mechanical response of the tissue (e.g. movement and/or force caused by the tissue). The actuator 16 may be passive (e.g. not be driven) during this time.

Data based on this sensor signal (first movement data) may be stored by the controller 36, and may indicate the response of the tissue to stimulus, e.g. the tissue response in the absence of active actuator movement.

The same electrical stimulus can then be applied 110 again, and this time the mounting is moved by the actuator 16 according to the first movement data. At this stage repeated cycles of the stimulus may simply be applied with concurrent (e.g. synchronised) actuator movement based on the first movement data so that data indicating the tissue response can be gathered.

As an optional further refinement of this approach, a second data acquisition cycle 108 may be performed subsequent to the acquisition of the first movement data. Thus, while the same electrical stimulus is being applied 108 again, and the mounting (and/or the rod) is being moved by the actuator 16 according to the first movement data, second movement data can be acquired from the sensor 18. This can indicate the response of the tissue 44 to stimulus in the absence of load, such as that due to the inertia of the actuator 16 and/or sensor 18 and/or mounting.

An experimental protocol for the study of the tissue sample 44 may include repeated, cyclic, applications 110 of the electrical stimulus with data collection 112. This may be performed repeatedly, e.g. periodically, for some defined period or until some end condition is fulfilled 114, such as a number of cycles of stimulus and response. Accordingly, the electric stimulus may be applied repeatedly and during each application 110 of the electrical stimulus and the tissues response, the actuator 16 can be controlled to move based on the first movement data and the second movement data.

The first movement data and the second movement data may comprise force data—e.g. the sensor 18 may comprise a force transducer. In these embodiments the controller 36 may be configured to predict the motion of the tissue sample 44 based on the force that it generates. The model used to perform this prediction may be based on the so-called Windkessel approach as described in the American Journal of Physiology: Heart and Circulatory Physiology, Volume 265 Issue 3, September 1993, Pages H899-H909 “Impact of ejection on magnitude and time course of ventricular pressure-generating capacity” D. Burkoff et. Al. Other approaches may be used. Whichever method is chosen—this mechanical feedback based approach may allow for synchronisation between force generation and movement−similarly to what would happen in 3D in vivo (pressure and volume).

At the end of the experiment (e.g. once the selected duration is complete, or the end condition has been met), the experimenter may wish to investigate the response of the tissue to mechanical protocols (e.g. measure force while tissue is progressively stretched). This “end-point assay” can be done manually or automatically. In the manual mode data acquisition is enabled while the user controls the position of the actuator (+/− direction). In the automatic mode (automatic end-point assay) the user inputs the desired position values and the system automatically stretches the tissue to these positions while simultaneously acquiring movement data from the sensor. The movement data may comprise position and force data.

In this mode, the user may select:
a) upon reaching a given position how long to wait before moving on to the next position (timer target time), and
b) how fast should position change from current to next (steepness). For example, the user can select to stay in a given position for 2 minutes. And once target time has been reached (e.g. =2 minutes) the controller 36 may move the actuator to the next position, which it may hold for a selected period (e.g. 30 s). This process may repeat itself until all positions have been reached. While the actuator is moving through the different positions the system may simultaneously acquire, analyze, and display data. When each desired position is reached the user can select between:
No actuator movement (tissue contracts with no changes in movement)
Waveform mode

This enables waveforms to be actuated on the tissue at different degrees of stretch. In “waveform mode” the actuator may be controlled to move in response to the electrical stimulus as described above.

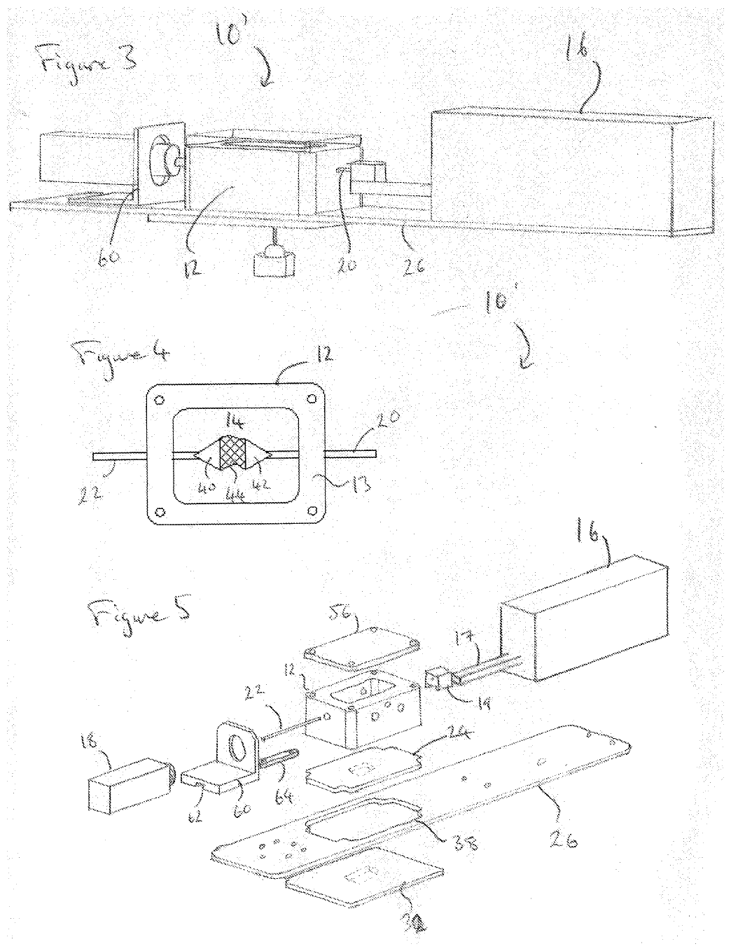

FIG. 3, FIG. 4, and FIG. 5 illustrate an example of the bioreactor apparatus 10′ including a number of optional features. In addition to the features of the bioreactor apparatus 10 described above with reference to FIG. 1 and FIG. 2.

A significant feature of the example shown in FIGS. 3 to 5 is the construction of the bioreactor vessel. It can be seen that the vessel 12 comprises a container 14 having thermally insulating walls 13, and a thermally conductive base plate 24 (more conductive than the walls 13). The base plate 24 is secured to the walls to provide a container 14 for holding liquid. The walls 13 comprise apertures which may be threaded or comprise other releasable disconnect features to allow sealing engagement with fluid lines for the supply and/or exhaust of fluids such as liquids and gas (e.g. O₂) as explained above. The bioreactor vessel 12 may also comprise a light transmissive lid 56, which may be transparent. This lid can comprise a transparent material such as glass or polycarbonate to allow visual inspection of the tissue sample 44 during experiments.

The lid may comprise a set of holes to allow the lid to be screwed or otherwise removably affixed to the container. The upper edge (top surface) of the container 14 walls may comprise a sealing feature such as a groove and/or gasket. Such features can be used to improve the seal between the lid and the container.

The container 14 may comprise one or more optical windows, such as that provide by the light transmissive lid, to allow radiation to be directed onto the tissue sample 44, and optionally also to sense the interaction of the tissue sample 44 with such radiation. This can allow investigation of the tissue sample 44 during experiments by methods such as microscopy, spectroscopy, and diffraction. In the example illustrated in FIGS. 3 to 5, a laser may be arranged beneath the chassis 26 (e.g. under the seat 38) and arranged to direct laser light up through a window in the seat 38. A corresponding window in the base 24 of the container 14 is provided to allow the laser light to be provided to the tissue sample 44.

It can be seen in FIGS. 3 to 5 that the apparatus 10′ may comprise a rigid chassis 26 such as a plate, to which the sensor 18 and actuator 16 can be fixed, so that the reactor vessel 12 can be mounted between them. The chassis plate may comprise a recess 38, such as a portion of the chassis plate 26 which has been cut out. The base 24 of the bioreactor vessel 12 may also comprise a plate 24, which may have the same form as the recess 38 in the chassis plate.

The sensor 18 and/or the actuator may be adjustable, to enable the neutral (e.g. starting) position of the actuator rod 20 and/or the sensor rod 22 to be moved toward or away from the reactor vessel. This can be accomplished in a variety of ways. For example, the sensor 18 may be connected to a slidable base 60. The slidable base 60 may have an elongate groove 62 on its under-side, and a rail 64 which fits in the groove may be fixed to the chassis 26 beneath the slidable base 60. The rail 64 and the groove 62 may be aligned with the direction of action of the actuator, so that the sensor 18 can be moved by sliding the base 60 along the rail 64 towards and away from the bioreactor vessel 12 without causing misalignment of sensor 18 and actuator 16.

Another way to provide adjustability is illustrated with respect to the actuator 16 shown in FIGS. 3 to 5. As shown the actuator 16 comprises an arm 17, to which the actuator rod 20 is connected by a collar 19, which sits on the arm 17. The collar 19 is able to slide longitudinally along the arm 17 to allow for longitudinal adjustment of the actuator rod 20 position. The collar 19 may partially or completely surround the arm to constrain the transverse position of the collar thereby to reduce the risk of misalignment. The collar 19 and/or the slidable base if used may be fixed in position using a grub screw or similar means to hold them in a fixed position once they have been adjusted. It will be appreciated however that many of the details set forth herein, whilst they may have particular application to some of the many technical problems described herein need not be provided in all embodiments, and no inference of essentiality is to be drawn from the application of one or other feature to one or other technical problem. Other technical problems may be addressed.

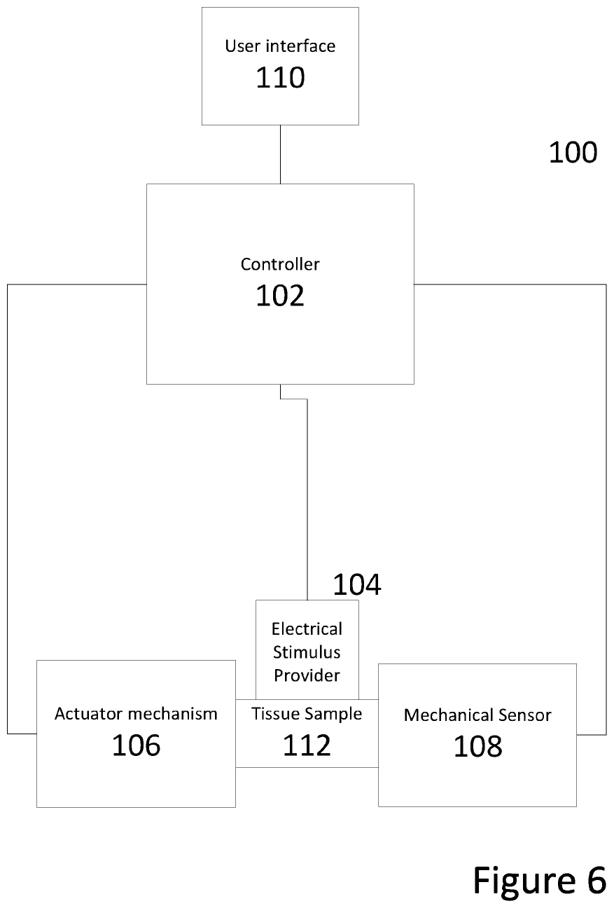

FIG. 6 gives a schematic view of a bioreactor apparatus 100 such as any one of those described above.

The bioreactor apparatus 100 comprises a controller 102 connected to, and operable to communicate with and control an electrical stimulus provider 104, an actuator mechanism 106, and a mechanical sensor 108. In particular, the controller is configured to provide instruction signals to the electrical stimulus provider 104 and the actuator 106, and to obtain measurement signals from the mechanical sensor 108. The controller may also be able to provide instructions to the mechanical sensor 108 and also to obtain information from the electrical stimulus provider 104 and the actuator mechanism 106.

The controller 104 is also connected to and operable to communicate with a user interface 110. In particular the controller may be configured to obtain instruction signals from the user interface 110. These instruction signals may relate to the operation of the actuator mechanism 106 and/or the electrical stimulus provider 104. The controller may be configured to modify these obtained signals and/or to provide instructions to the actuator 106 and/or electrical stimulus provider 104 based on them.

As described in more detail below, during operation of the bioreactor apparatus 100 the controller 102 instructs the electrical stimulus provider to provide an electrical stimulus to a sample of cardiac tissue 112, to define an electrical stimulus waveform. The electrical stimulus waveform may be provided as a series of periodic pulses.

The controller 102 also instructs the actuator mechanism 108 to provide mechanical force to the sample of cardiac tissue 112. In particular, the instruction signal provided from the controller 102 to the actuator mechanism 108 may be a simple “ON” or “trigger” signal. In response to receiving such a signal, the actuator mechanism 108 may extend and compress the tissue to define a mechanical force waveform. The mechanical force waveform that is outputted is based on a motion profile has been determined e.g. at the controller and provided to the actuator mechanism 108 where it is stored in a memory of the actuator mechanism, e.g. a volatile memory.

Such a motion profile may be calculated using a model (such as a Windkessel model) as is described in more detail below. Each of the mechanical and electrical waveforms that are provided to the cardiac tissue sample 112 are periodic and attempt to mimic, in vitro, the beating of an in vivo heart. As is described in more detail below, during operation of the apparatus, the parameters of the model can be updated by the controller based on input received from the user interface. In response, the controller is arranged to provide an updated motion profile to the actuator for it to provide to the tissue. Such an adjustment may be provided in real time, while the apparatus is in operation. As such, real time control of the actuator position may be provided.

The controller 102 is also configured to obtain a measurement signal from the mechanical sensor 108, to determine the response of the tissue to one or both of the electrical stimulus signal and mechanical force waveform.

The user interface 110 may be provided by a computer terminal, and is operable to enable a user to set and or modify the time period of the electrical stimulus waveform provided to the tissue sample 112, for example by selecting and adjusting the time period of the (e.g. the frequency of electrical pulses). The controller 102 is configured to provide an instruction signal to the electrical stimulus provider 104 based on the user input from the user interface 110, and as is described in greater detail below, the controller is configured to provide an instruction signal to the actuator mechanism 106, for example to modify that signal, based on the user input from the user interface 110.

The electrical stimulus provider 104 the actuator mechanism 106, tissue sample 112, electrical stimulus provider 104 and mechanical sensor 108 may all be physically arranged as described above with reference to FIGS. 1 to 5. The actuator mechanism 106 applies force to the tissue sample 112 in the same way as described above, e.g. via the use of an actuator rod. The electrical stimulus provider 104 and mechanical sensor 108 may also be configured to operate as described in FIGS. 1 to 5.

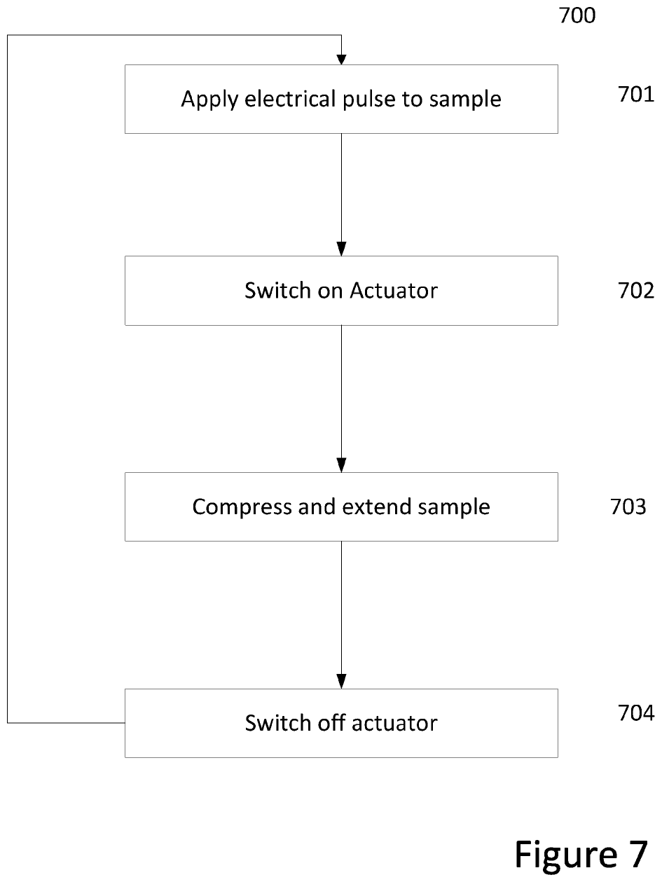

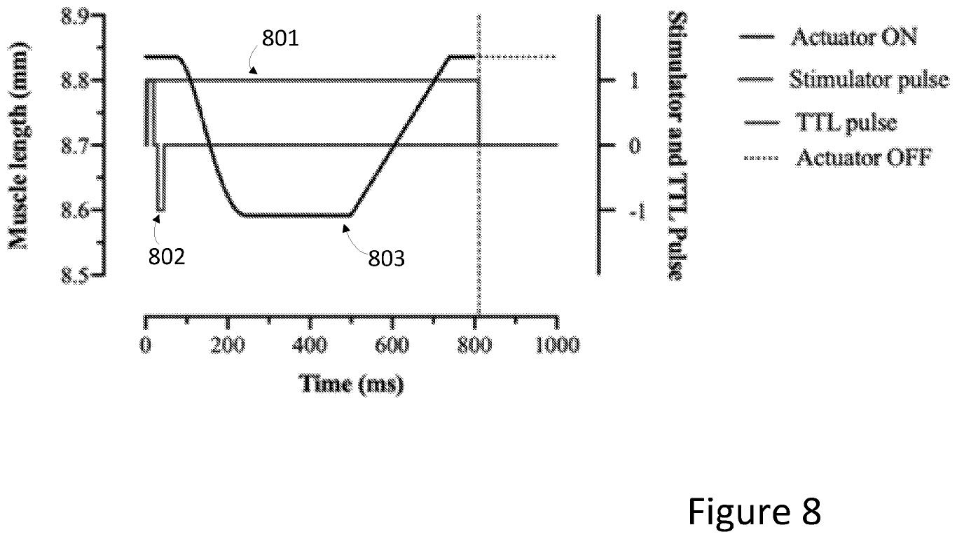

FIGS. 7 & 8 illustrate an example method 700 that can be performed by the bioreactor apparatus 100. The method 700 is cyclical and aims to provide an in vitro simulation of a beating heart. FIG. 7 shows the steps of the method in the form of a flow chart, while FIG. 8 shows a graph which illustrates, over the course of a single cycle: the instruction signal provided to the actuator 801, the electrical stimulus signal 802 provided to the tissue sample, and an indication 803 of how the length of cardiac tissue varies over the course of one cycle of the method. Because the tissue sample is shortened and lengthened by the operation of the actuator, the indication of tissue length over the cycle indicated by line 803 also corresponds to the movement of the actuator, e.g. the actuator rod, over the course of the cycle.

In a first step 701 of the method 700, the electrical stimulus provider, in response to an instruction signal from the controller, provides an electrical stimulus waveform, e.g. in the form of an electrical pulse, to the sample of cardiac tissue. As shown by line 802 in FIG. 8, the electrical stimulus waveform is in the form of a bipolar pulse, which lasts in the order of milliseconds to 10 s of milliseconds, for example between 4 milliseconds and 20 seconds. The length of time of the bipolar pulse may itself be adjustable, e.g. by a user via the user interface, for example to a value in this range. In some examples the user interface may be configured to enable the user to set the value at set points, e.g. in 2 ms increments, within the range.

As shown in FIG. 8, at the same time as the initiation of the electrical stimulus pulse, an instruction signal 801 is provided by the controller to the actuator instructing it to switch on. This step is illustrated as 702 in FIG. 7 but it will be appreciated that it may be performed simultaneously with the application of the electrical pulse 701. The instruction signal is provided in the form of a Transistor-Transistor Logic digital line being switched into an ON state, as shown in FIG. 8. Upon receiving the instruction signal the actuator is configured to output a mechanical force waveform, based on a calculated motion profile, as described elsewhere herein. Specifically, as illustrated by line 803 the actuator begins to operate so as to shorten and then extend of the cardiac tissue sample 703, for example by moving the distance between the mountings that hold each edge of the tissue sample, as described above. As illustrated in FIG. 8, the motion profile upon which the actuator movement is based may provide for some delay between the time the actuator is switched on and the time that the actuator begins to move to shorten the tissue sample.

As shown by line 803, based on the motion profile, the actuator acts to reduce the length of the tissue so as to move the tissue from an extended state to a shortened state. The extent and rate of compression may be calculated by the controller using a Windkessel model, discussed in more detail below. This can predict, for the properties of a given tissue sample, and the force produced by the tissue sample in response to the electrical stimulus, the extent and rate at which the tissue would shorten in vivo, and provide that profile to the actuator. These parameters can be inputted and modified by a user, e.g. at the user interface, and the controller can update the model based on the inputted values.

The model can predict, for a given force produced by the tissue sample in response to the electrical stimulus (e.g. the measured by the mechanical sensor) the corresponding change in the length of the tissue that should accompany this force. As just one example, in the event that the tissue sample produces a 10 N of force and the Windkessel model predicts a 0.2 mm change in length for this amount of force, if the parameters of the model (e.g. relating to the properties of the tissue) are modified by a user, the prediction provided by the model may change to a 0.5 mm shortening of tissue length for the same amount of force. This change in prediction can happen in real-time. However, the corresponding update to the motion profile provided to the actuator may also occur after some delay, without affecting the results of the study, as is discussed in more detail further below.

Once the tissue is shortened to the extent predicted by the model, the actuator then holds the tissue in this shortened state for a period of time before extending the tissue back into the extended state.

Once this motion has been completed a further instruction signal is provided from the controller to the actuator, in the form of the Transistor-Transistor Logic digital line being switched into an OFF state, which thereby switches off the actuator 704. The actuator is then dormant until it receives a further ON instruction signal simultaneously with the next electrical pulse 701, and is switched back on to perform another cycle of compression and extension of the tissue.

As such, a periodic mechanical force waveform is defined (illustrated by line 803), comprising a static interval, during which the actuator remains in the extended state, and a dynamic interval during which the sample is moved into the shortened state and moved back into the extended state. A periodic electrical stimulus waveform is also defined, comprising a series of repeating pulses. Due to the actuator switching on at the same time as the application of the electrical pulse each cycle, and providing a full single cycle of compression and extension of the tissue between two electrical pulses, the mechanical force waveform can be considered to be synchronised with the electrical stimulus waveform.

Each cycle can also be subdivided based on the analogous phases of a heartbeat. Specifically, with reference again to FIG. 8, the interval in which the tissue is shortened/compressed corresponds to the systolic phase.

The remainder of each cycle corresponds to the diastolic phase, which itself can be further subdivided. Specifically, the period in which the tissue is held stationary in the shortened state corresponds to an “isovolumetric relaxation” phase in which the tissue relaxes without changes in length. The period in which the tissue extends back to the extended state corresponds to a “rapid filling phase”, and the period in which the tissue is held stationary in the extended state corresponds to a “diastasis” phase, in which filling is less rapid and volume is held constant in the extended state prior the next contraction.

FIG. 8 shows an example of a single waveform cycle, in which the period of the two waveforms is 1000 ms, and the actuator is on for a period of approximately 811 ms. However, it will be appreciated that this is merely exemplary. In order to effectively model the response of cardiac tissue at a range of different heart rates, for example to model the effects of elevated heart rate (tachycardia) the frequency of electrical pulses can be adjusted, for example by a user via the user interface. In doing so, the period of the electrical stimulus waveform is adjusted. In these circumstances the mechanical force waveform must also be correspondingly adjusted in response to the change in the period of the electrical waveform, in order for the compression and extension of the tissue to continue to fit between each electrical pulse or heartbeat.

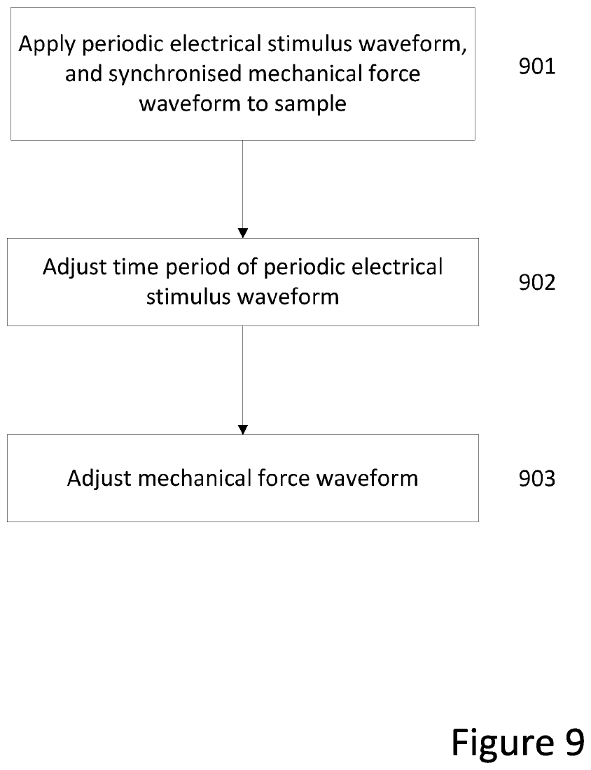

An overview of this method 900 is shown in FIG. 9. In a first step 901 an electrical stimulus waveform with an initial time period, and a synchronised mechanical force waveform, are applied to the tissue sample as described above with reference to FIGS. 7 and 8. The time period of the electrical stimulus waveform, e.g. the frequency of electrical pulses, is then adjusted 902. This adjustment may be provided in response to an instruction from a user via the user interface, for example a user may input a desired time period/frequency of pulses at a computer terminal or other device. In particular the controller may receive an indication of the instructed frequency from the user interface then instruct the electrical stimulus provider to provide electrical pulses at that frequency.

In response to the adjustment to the time period of the electrical stimulus waveform, the mechanical force waveform is correspondingly adjusted 903.

The adjustment of the mechanical force waveform may be provided in real-time while the apparatus is running, e.g. for the next cycle. However, in other examples there may be some delay between the adjustment of parameters by the user (e.g. the frequency of the electrical pulses, or the other parameters of the Windkessel model) and the adjustment provided by the mechanical force waveform, e.g. a delay of approximately 5 seconds. However, as the study of the tissue response at a particular set of conditions takes place over much longer timescales (in the order of days), and as there is very little beat-to-beat variability to the response of the tissue sample, this delay will not have any noticeable effect on the overall measured tissue response.

The controller is configured to update the model used to provide the mechanical force waveform, and to provide an updated motion profile to the actuator, in response to an instructed change in electrical stimulus pulse frequency. The mechanical force waveform can be modified in different ways. Preferentially, in the case were the electrical stimulus waveform period is reduced (e.g. the frequency of electrical pulses is increased) the time that the actuator is switched off for can be reduced, that is, the actuator simply switches on more quickly after it was switched off, in line with the faster electrical pulses, and outputs the same motion profile while it is in the ON state. Because the actuator off time corresponds to the diastasis phase of the heartbeat, in such examples this phase is shortened. However, in other examples, such as when the electrical stimulus period is reduced more than the existing off time of the actuator, the motion profile that is outputted by the actuator is itself shortened. For example, the delay between the actuator switching on and the actuator starting to shorten the tissue sample may be reduced. In some examples, the time taken to extend the tissue back into the extended state, corresponding to the “rapid filling”, phase can also be shortened by increasing the rate of this extension. The time taken to shorten the tissue (systole) may also be reduced, for example by increasing the rate at which the tissue is shortened. Also, the time the tissue is held in the shortened state (isovolumetric filling phase) can also be shortened in some examples. The various adjustments discussed above can be provided in any combination, and can also be provided on instructions received from the user.

Throughout the methods described, the response of the tissue is measured using the mechanical sensor. As such, in addition to the adjustments to the electrical stimulus waveform and the mechanical force waveform described above in relation to FIGS. 7 to 9, both waveforms may also be adjusted based on the mechanical feedback measured by the sensor, as has been described above.

Any feature of any one of the examples disclosed herein may be combined with any selected features of any of the other examples described herein. For example, features of methods may be implemented in suitably configured hardware, and the configuration of the specific hardware described herein may be employed in methods implemented using other hardware. It will be appreciated for example that the actuator mechanism 106 described in relation to FIG. 6 may comprise an actuator 16 and actuator rod 20 as are described with reference to FIGS. 1 to 5. Furthermore, it will be understood that the tissue sample 112 of the embodiment of FIG. 6 may be mounted and held in position on the apparatus 100 in the same way as the sample described in relation to FIGS. 1 to 5. It will also be appreciated that the bioreactor apparatus 100 may also comprise some or all of the other features of the bioreactor apparatus 10, 10′ as described in relation to FIGS. 1 to 5, but which are not shown in the simplified schematic diagram of FIG. 6.

It will be appreciated from the discussion above that the embodiments shown in the Figures are merely exemplary, and include features which may be generalised, removed or replaced as described herein and as set out in the claims.

As a first example, the liquid system mentioned above is described as having a flexible wall portion and being thermally insulated. This may be achieved in a any of a variety of ways—for example some or all of the whole tube may be flexible and may be surrounded by thermal insulation. A gap in the insulation may be provided to provide the flexible wall portion for pumping the liquid.

As a second example, in description of the actuator rod 20 and the sensor rod 22 with reference to FIG. 1, the possibility of a dog-leg is mentioned. It will however be appreciated in the context of the present disclosure that an equivalent effect may be achieved by arranging the apertures and/or the sensor and actuator so that the rods can be at an oblique angle. This or any other non-horizontal connection between mounting and sensor/actuator can be used, provided that the relevant apertures are disposed above the level of the tissue sample 44 on the mounting (e.g. above the required liquid level).

As a third example, the linear actuator may be operable to push or pull the first part 42 of the mounting towards and away from the actuator according to any of a variety of control schemes. For example it may provide movement of the actuator according to a waveform motion profile. The motion profile of the actuator may define changes in the position of the actuator as a function of time. For example, a linear movement would mean a step change (e.g. 0.1 mm) per unit time (e.g. every 1 s), and it would be a straight line if visualised in an XY-graph (where X=time, Y=position). The controller may operate the actuator according to a motion profile such as any one or more of the following:
Windkessel model, which may comprise waveform movement in a series of steps. A first step may allow the sample tissue to shorten (e.g. contract). A second step may follow in which the actuator is not driven to move (e.g. held stationary) for a time interval, which may provide no actuator movement. A third step may comprise a period of stretching the tissue, e.g. back to its pre-contraction length at the outset of the first step (e.g. just before the non-linear motion profile was applied). This, sequence may be repeated to provide cyclic shortening and re-stretching of the tissue. The stretching and contracting steps may be linear (e.g. constant speed).
A Force-clamp mode in which a linear motion profile begins when the sensor indicates that the force generated by the tissue reaches a selected level (e.g. exceeds a threshold level or drops below a minimum level). For example, the controller may be configured to move to allow the tissue to shorten when the force generated by the tissue reaches a selected fraction of the maximum force (e.g. 50% of maximum force). The maximum force may be based on force data sampled during at least one previous contraction cycle of the tissue (e.g. the immediately preceding cycle, or an average over previous cycles)
Custom modes in which the controller may operate the actuator according to numerically-defined motion waveforms such as a sinusoid, a square wave, a step or Heaviside, and impulse move (rapid alternation between +/−).

The controller 36 may be configured to provide a selected delay between the mechanical action (movement of the actuator) and electrical stimulus applied to the tissue. Thus the stimulus and mechanical response of the actuator may be desynchronized to mimic electromechanical mismatch as seen in disease states, etc.

The actuator may also be operated by the controller 36 automatically to run an end-point assay.

It will be appreciated by the skilled addressee in the context of the present disclosure that the “Windkessel Model” may be used to predict changes in tissue length with time as a function of Rc, Ca, Ra, and force generated by tissue; where
Rc=peripheral resistance
Ca=arterial compliance
Ra=arterial impedance

The controller may be configured to use these parameters and the Windkessel Model to determine a waveform (movement profile) for the actuator. The controller may be configured to obtain these parameters from memory, or from a user input. Examples of Windkessel models of cardiac tissue may be based on the definitions provided in Westerhof N, Lankhaar J W, Westerhof BE (February 2009). “The arterial Windkessel”. Medical & Biological Engineering & Computing. 47 (2): 131-41. And Cappello A, Gnudi G, Lamberti C (March 1995). “Identification of the three-element windkessel model incorporating a pressure-dependent compliance”. Annals of Biomedical Engineering. 23 (2): 164-77.

As a fourth example, the chassis plate has been described as including a recess for insertion of the base 24 of the bioreactor vessel 12 to seat the vessel in position. It will be appreciated however that any appropriate complementary features may be used. For example, the chassis may carry a protrusion, and the base 24 of the bioreactor vessel 12 may be recessed so that the protrusion can fit into the recess. Other arrangements of complementary engagement features are possible, but typically the two fit together snugly so as to ensure that the position of the bioreactor vessel 12 can be fixed to allow mechanical, electrical, and fluid connections to the other parts of the apparatus 10′. In similar vein, the slidable base to which the sensor 18 shown in FIGS. 3 to 5 is fixed may comprise an elongate rail for fitting in a groove disposed on the base. It doesn't matter whether the groove or rail is provided on either or both element, provided that transverse movement of the slidable base is constrained whilst longitudinal movement (towards/away from the bioreactor vessel) is permitted. This can provide easy adjustment of the apparatus 10′ (e.g. when a smaller or larger tissue sample 44 is used) without risking misalignment between sensor 18 and actuator. This arrangement of using a slidable base has been shown and described with reference to the sensor, but it will be appreciated in the context of the present disclosure that the actuator may be mounted in the same way to provide adjustability of the actuator.

With reference to the drawings in general, it will be appreciated that schematic functional block diagrams are used to indicate functionality of systems and apparatus 10, 10′, 100 described herein. It will be appreciated however that the functionality need not be divided in this way, and should not be taken to imply any particular structure of hardware other than that described and claimed below. The function of one or more of the elements shown in the drawings may be further subdivided, and/or distributed throughout apparatus of the disclosure. In some embodiments the function of one or more elements shown in the drawings may be integrated into a single functional unit.

In some examples the functionality of the controller 36, 102 may be provided by a general purpose processor, which may be configured to perform a method according to any one of those described herein. In some examples the controller 36, 102 may comprise digital logic, such as field programmable gate arrays, FPGA, application specific integrated circuits, ASIC, a digital signal processor, DSP, or by any other appropriate hardware such as a general purpose computer. In some examples, one or more memory elements can store data and/or program instructions used to implement the operations described herein. Embodiments of the disclosure provide tangible, non-transitory storage media comprising program instructions operable to program a processor to perform any one or more of the methods described and/or claimed herein and/or to provide data processing apparatus as described and/or claimed herein. The controller 36, 102 may comprise an analogue control circuit which provides at least a part of this control functionality. An embodiment provides an analogue control circuit configured to perform any one or more of the methods described herein.

The above embodiments are to be understood as illustrative examples. Further embodiments are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. An apparatus comprising:
an actuator for moving an actuator rod;
a bioreactor vessel comprising:
a container for holding a liquid;
a mounting for mounting a tissue sample in the container;
and, an actuator coupling to enable the actuator rod to be connected for applying mechanical force to the tissue sample;
a seat, fixed with respect to the actuator and configured for locating the reactor vessel in a location selected so that the actuator can be connected for applying said force via the actuator coupling,
wherein the reactor vessel is removable from the apparatus.

2. The apparatus of claim 1 comprising a mechanical force sensor arranged for sensing mechanical force generated by the tissue sample.

3. The apparatus of claim 2 comprising a controller configured to sense said mechanical force, and to control the actuator based on the sensing.

4. The apparatus of claim 3, wherein the apparatus is configured to provide a cyclic stimulus to the tissue sample wherein the control of the actuator during a cycle of the stimulus is based on sensing performed in a preceding cycle of the stimulus.

5. The apparatus of any preceding claim wherein the reactor vessel comprises a gas inlet, and the apparatus is configured to supply gas to the inlet to maintain a positive gas pressure in a headspace of the container above the liquid.

6. The apparatus of any preceding claim, wherein the seat comprises a heat provider and the reactor vessel comprises a thermally conductive base.

7. The apparatus of claim 6 wherein the heat provider is configured to control the heat provided to the thermally conductive base based on a temperature of the liquid.

8. The apparatus of claim 7 comprising a sensor for sensing the temperature of the liquid in a liquid recirculation system of the apparatus.

9. The apparatus of any preceding claim, wherein the container comprises an inlet for flow of liquid into the container, and an outlet for the flow of liquid out of the container.

10. The apparatus of any preceding claim wherein the actuator coupling comprises an aperture arranged so that, when the reactor vessel is located in said location in the seat, an actuator rod can connect the actuator to the tissue sample.

11. The apparatus of claim 10 further comprising the actuator rod.

12. The apparatus of claim 11 wherein the rod is removable from the actuator, for example wherein the rod is adapted for disinfection in an auto-clave.

13. The apparatus of any preceding claim wherein the reactor vessel is adapted for disinfection in an auto-clave.

14. The apparatus of any preceding claim wherein the container is thermally insulating.

15. The apparatus of any preceding claim wherein the reactor vessel comprises at least one of a sensor and an electrical stimulus provider for providing electrical stimulus to the tissue sample.

16. The apparatus of claim 15 wherein the apparatus comprises a signal interface for connection to the at least one of a sensor and an electrical stimulus provider, and the seat is arranged so that when the vessel is located in said seat it is positioned for connection of the signal interface to a corresponding interface of the bioreactor vessel.

17. The apparatus of any preceding claim wherein the seat and the reactor vessel comprise complementary engagement features for locating the reactor vessel in said location.

18. A method of providing an in vitro cardiac model, the method comprising:
providing a stimulus to a tissue sample of cardiac tissue to cause a mechanical response of the cardiac tissue;
sensing said mechanical response; and
subsequently providing the stimulus to the tissue sample while providing mechanical feedback to the tissue sample, wherein the mechanical feedback is based on the sensed mechanical response.

19. The method of claim 18 wherein the mechanical feedback is synchronised with the stimulus.

20. The method of claim 18 or 19 wherein the stimulus is provided by an interval of a cyclic waveform and the mechanical response is sensed during a first interval and the mechanical feedback is provided during a second, subsequent interval.

21. The method of claim 20 wherein the interval comprises at least one cycle of the cyclic waveform.

22. The method of any of claims 18 to 21, wherein the tissue sample is mounted in the container of the apparatus of any of claims 1 to 17.

23. The method of claim 22, wherein the mechanical feedback is provided by the actuator.

24. The method of claim 23 or 22 wherein the actuator is configured to perform said sensing of the mechanical response.

25. The method of any of claims 18 to 24 wherein the stimulus is provided to the tissue via the mounting.

26. A method of providing an in vitro cardiac model, the method comprising:
applying a periodic electrical stimulus waveform to a sample of cardiac tissue; and
applying a periodic mechanical force waveform to the sample;
wherein the mechanical force waveform is synchronised with the electrical stimulus waveform;
the method further comprising:
adjusting the time period of the periodic electrical stimulus waveform; and
adjusting the mechanical force waveform based on the adjustment to the time period of the electrical stimulus waveform.

27. The method of claim 26, wherein applying the periodic mechanical force waveform comprises cyclically moving sample of cardiac tissue between a shortened state and an extended state.

28. The method of claim 27, wherein each period of the mechanical force waveform comprises a static interval in which the sample is held static in the extended state, and a dynamic interval in which the sample is moved into the shortened state and moved back into the extended state.

29. The method of claim 28, wherein adjusting the mechanical force waveform comprises adjusting at least one of:
the duration of the static interval; and
the duration of the dynamic interval.

30. The method of any of claims 26 to 29, comprising obtaining an instruction signal and providing the adjustment to the period of the electrical stimulus waveform in response to the obtained instruction signal.

31. The method of claim 30, wherein the instruction signal is obtained from a user interface.

32. The method of any of claims 26 to 31, wherein the electrical stimulus is provided by a periodic electrical pulse.

33. The method of claim 32, wherein the periodic electrical pulse is bipolar.

34. The method of any of claims 26 to 33, further comprising providing a trigger signal simultaneously upon each application of the electrical stimulus, and applying the mechanical force in response to the trigger signal.

35. The method of any of claims 26 to 34, wherein applying a periodic mechanical force waveform to the sample comprises providing instructions to an actuator to shorten and/or extend the sample.

36. The method of any of claims 26 to 35, further comprising sensing a mechanical response of the sample.

37. The method of claim 36, further comprising adjusting at least one of:
periodic electrical stimulus waveform; and
periodic mechanical force waveform
based on the sensed response.

38. The method of any of claims 26 to 37, wherein the mechanical force waveform being synchronised with the electrical stimulus waveform comprises the start of each cycle of the periodic mechanical force waveform having a constant phase and/or timing offset from the start of each cycle of the periodic electrical stimulus waveform.

39. A controller configured to perform the method of any of claims 18 to 38.

40. A computer program product comprising computer program instructions configured to program a controller to perform the method of any of claims 18 to 38.
